(19) United States
(12) Patent Application Publication
(10) Pub. No.: US 2026/0369082 A1
(43) Pub. Date: Nov. 19, 2026

(54) INTEGRATED MOBILE SYSTEM FOR FORMATION ROCK ANALYSIS

(71) Applicant: SCHLUMBERGER TECHNOLOGY CORPORATION, Sugar Land, TX (US)

(72) Inventors: Tetsushi YAMADA, Cambridge, MA (US); Romain PRIOUL, Cambridge, MA (US); Karim BONDABOU, Clamart (FR); Ravi TEJWANI, Cambridge, MA (US)

(21) Appl. No.: 19/138,691

(22) PCT Filed: Nov. 30, 2023
(86) PCT No.: PCT/US2023/081725

§ 371 Date: Jun. 13, 2025

(30) Foreign Application Priority Data
Dec. 14, 2022 (EP) 22306870.1

(51) Int. Cl. G06V 20/60; G06T 7/11; G06T 7/50; G06T 7/64; G06V 10/26; G06V 10/46; G06V 10/764; G06V 10/94; G06V 20/50; G06V 20/70

(52) U.S. Cl. CPC G06V 20/60; G06T 7/11; G06T 7/50; G06T 7/64; G06V 10/26; G06V 10/46; G06V 10/764; G06V 10/94; G06V 20/50; G06V 20/70; G06T 2207/20084; G06T 2207/30181

(57) ABSTRACT

A method for classifying cavings particles includes using a mobile device to acquire a digital image of rock particles generated while drilling a subterranean wellbore. The digital images may be processed to generate a segmented image that identifies individual ones of the rock particles and to extract a plurality of geometry properties from selected ones of the identified rock particles depicted in the segmented image. The plurality of geometry properties includes size features and shape features. At least one of the size features may be evaluated to identify cavings particles among the identified rock particles. A cavings particle shape classification may be generated by evaluating a plurality of the shape features to label each of the identified cavings particles with a corresponding cavings shape.

### CROSS REFERENCE TO RELATED APPLICATIONS

This application claims the benefit of E.P. application Ser. No. 22/306,870.1, entitled “INTEGRATED MOBILE SYSTEM FOR FORMATION ROCK ANALYSIS,” filed Dec. 14, 2022, the disclosure of which is hereby incorporated herein by reference.

### BACKGROUND

Rock particles are produced during drilling operations for oil and gas exploration and recovery, geothermal, and scientific exploration. These rock particles are commonly classified as either cuttings or cavings. Cuttings particles are those that are generated directly by the cutting action of the drill bit as it breaks the formation rock. Cavings particles are those that are not generated directly by the cutting action of the drill bit and are commonly particles that spall away from unstable sections of the wellbore.

It will be appreciated that rock particles generated during drilling (e.g., cuttings and cavings particles) are abundant in volume and number and may provide one of the lowest cost and most abundant data sources for understanding and characterizing the subsurface rock and formation properties. For example, the shape of cavings particles is sometimes used as an indicator of a formation rock failure mechanism. Moreover, the size and shape of cavings particles may be used to update a Mechanical Earth Model (MEM) of the subterranean formation including formation stress, pore pressure, and formation strength. In certain formation types, large cavings particles may be an indicator of an abnormal pressure zone or wellbore enlargement that may lead to loss of drilling fluid (circulation loss), stuck drill pipe, or even a partial collapse of the wellbore.

There is a need in the industry for improved methods for evaluating cavings particles, and particularly for methods that automate or partially automate the measurement process and thereby reduce human cost and shorten the turnaround time of the interpretation.

### BRIEF DESCRIPTION OF THE DRAWINGS

For a more complete understanding of the disclosed subject matter, and advantages thereof, reference is now made to the following descriptions taken in conjunction with the accompanying drawings, in which:

FIG. 1 depicts an example drilling rig including an example mobile system for evaluating rock particles obtained in a drilling operation.

FIGS. 2A and 2B (collectively FIG. 2) depict block diagrams of example mobile systems for evaluating cavings particles.

FIGS. 3A-3F (collectively FIG. 3) depict the operation of a mobile App employed by the mobile architecture shown on FIG. 2.

FIG. 4 depicts geometric relationships between a field of view, an angle of view, a camera focal length, and the height of a camera sensor.

FIG. 5 depicts a flow chart of an example method for evaluating cavings particles.

FIG. 6 depicts an example method for acquiring a digital image of cavings particles using a mobile device.

FIG. 7 depicts an example shape classification for cavings particles.

### DETAILED DESCRIPTION

Embodiments of this disclosure include systems and methods for evaluating cavings particles. One example method includes using a mobile device to acquire a digital image of rock particles generated while drilling a subterranean wellbore. The digital images may be processed to generate a segmented image that identifies individual ones of the rock particles and to extract a plurality of geometry properties from selected ones of the identified rock particles depicted in the segmented image. The plurality of geometry properties includes size features and shape features. At least one of the size features may be evaluated to identify cavings particles among the identified rock particles. A cavings particle shape classification may be generated by evaluating a plurality of the shape features to label each of the identified cavings particles with a corresponding cavings shape.

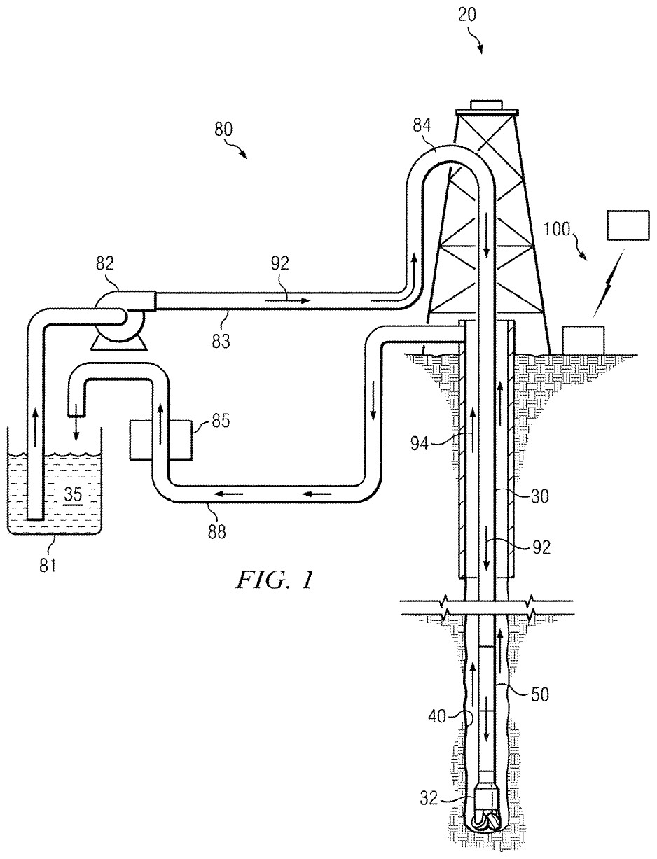

FIG. 1 depicts an example drilling rig 20 deployed at a wellsite that includes a mobile system 100 for evaluating cavings particles removed from circulating drilling fluid. The rig 20 may be positioned over a subterranean formation (not shown). The rig 20 may include, for example, a derrick and a hoisting apparatus (also not shown) for raising and lowering a drill string 30, which, as shown, extends into wellbore 40 and includes, for example, a drill bit 32 and one or more downhole measurement tools 50 (e.g., a logging while drilling tool and/or a measurement while drilling tool).

Drilling rig 20 further includes a surface system 80 for controlling the flow of drilling fluid used on the rig (e.g., used in drilling the wellbore 40). In the example rig depicted, drilling fluid 35 is pumped downhole (as depicted at 92) via a mud pump 82. The drilling fluid 35 may be pumped, for example, through a standpipe 83 and mud hose 84 in route to the drill string 30. The drilling fluid typically emerges from the drill string 30 at or near the drill bit 32 and creates an upward flow 94 of mud through the wellbore annulus (the annular space between the drill string and the wellbore wall). The drilling fluid then flows through a return conduit 88 and solids control equipment 85 (such as a shale shaker) to a mud pit 81. Drill cuttings created while drilling the well and cavings particles that spall of the surface of the wellbore may be transported to the surface in the upward flow 94 of drilling fluid and may be removed from the fluid at the solids control equipment 85. It will be appreciated that the terms drilling fluid and mud are used synonymously herein.

The wellsite may include a mobile system 100 configured to evaluate images of rock particles, for example, to determine the size and/or shape of the particles as described in greater detail herein. The system 100 may include a mobile device including a digital camera (such as a cell phone or tablet) located at the rig site and in communication with other computer processing equipment deployed at the rig site and/or offsite. The disclosed embodiments are not limited in this regard. The system 100 may include computer hardware and software configured to automatically or semi-automatically evaluate images of the rock particles. To perform these functions, the hardware may include one or more processors (e.g., microprocessors) which may be connected to one or more data storage devices (e.g., hard drives or solid state memory). As is known to those of ordinary skill, the processors may be further connected to a network, e.g., to receive the images from a networked camera system (not shown) or another computer system. It will, of course, be understood that the disclosed embodiments are not limited the use of or the configuration of any particular computer hardware and/or software.

While FIG. 1 depicts a land rig 20, it will be appreciated that the disclosed embodiments are equally well suited for land rigs or offshore rigs. As is known to those of ordinary skill, offshore rigs commonly include a platform deployed atop a riser that extends from the sea floor to the surface. The drill string extends downward from the platform, through the riser, and into the wellbore through a blowout preventer (BOP) located on the sea floor. The disclosed embodiments are not limited in these regards.

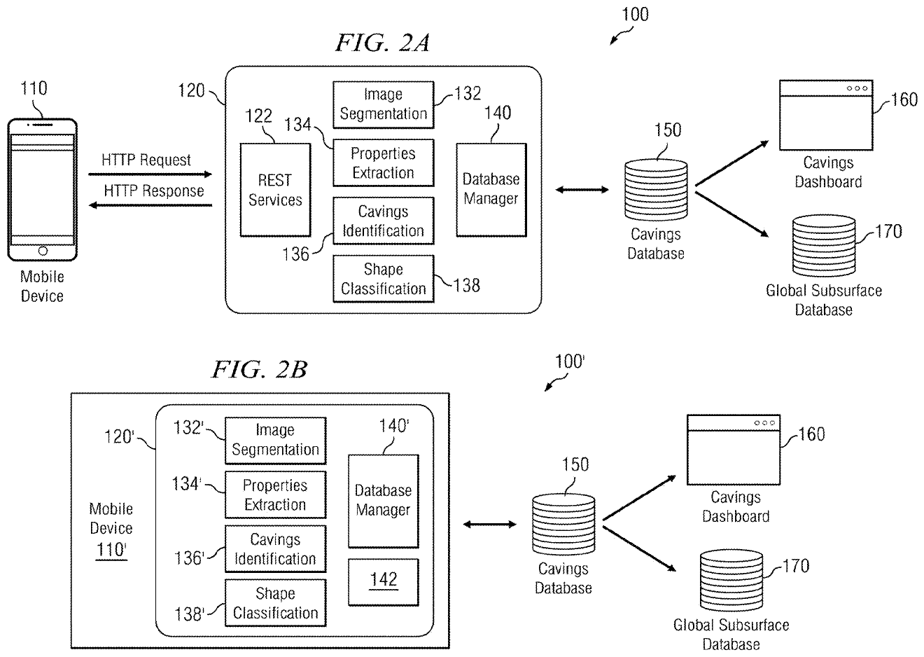

FIGS. 2A and 2B (collectively FIG. 2) depict example block diagrams of system 100 and system 100′ including a mobile architecture for evaluating rock particles obtained during a drilling operation. As shown in FIG. 2A, system 100 includes a mobile device 110 such as a smartphone or a tablet in mobile or wireless communication with a backend processing module 120 (e.g., via hypertext transfer protocol HTTP or via a standard local area network wireless connection). The mobile device 110 includes a digital camera that may be used, for example, by a mud logging engineer, a geoscientist, or other rig personnel to acquire images of rock particles (e.g., cuttings and/or cavings) removed from the circulating drilling fluid. The mobile device 110 further includes a software application (an App) configured to run on the mobile device and to guide the user through the process of acquiring and analyzing the digital images (as described in more detail below). For example, the App may be configured to send an acquired image to the backend module 120 for evaluation and data storage and to receive and display the evaluation results from the backend module 120.

With continued reference to FIG. 2A, the backend module 120 may include a Representational State Transfer (REST) module 122 for interfacing with the mobile device 110 via HTTP protocol (e.g., for receiving and an uploaded image from the mobile device and downloading processed data to the mobile device). The REST module may be configured to include web endpoints for evaluating and processing the received digital image. The backend module 120 may further include various processing modules for segmenting the image, extracting various particle properties from the image, and classifying the individual particles in the image. For example, in the depicted embodiment, the processing modules may include an image segmentation module 132 configured to process the digital image and generate a segmented image, an image properties extraction module 134 configured to extract properties from selected ones of the segmented particles, for example, including size and shape related features, a cavings identification module 136 configured to identify cavings particles as those having at least one size related feature greater than a threshold, and a shape classification module 138 configured to process the extracted properties and to group each of the identified cavings particles into a corresponding shape classification or category (as described in more detail below with respect to FIG. 5).

The backend module 120 may further include a database manager 140 configured to interface with a database 150 to store the raw image, the segmented image, and the properties and classification data generated by the processing modules. The system 100 may further include a dashboard 160 showing a listing of processed images stored in the database 150. In optional embodiments, the database 150 may be in further communication with a global subsurface database 170 that may include well log data for all the wells in a particular field or region.

It will be appreciated that the backend module 120, the database 150, the dashboard 160, and the global subsurface database 170 may be deployed on substantially any network server(s), for example including a cloud service such as Google Cloud Platform, Microsoft Azure, or an on-premises server located, for example, in the field or at a service provider. Moreover, the backend module may be deployed on substantially any networked personal computer or workstation. The disclosed embodiments are not limited to any particular server or hosting architecture.

In FIG. 2B, a processing module 120′ is deployed in the mobile device and is accessible to the App. For example, the App may be configured to access the module 120′ for image evaluation and temporary data storage and to receive and display the evaluation results. As described above with respect to FIG. 2A, module 120′ may include various processing modules for segmenting the image, extracting various particle properties, and classifying the individual particles in the image. For example, in the depicted embodiment, the onboard processing modules may include an image segmentation module 132′ configured to process the digital image and generate a segmented image, an image properties extraction module 134′ configured to extract properties from selected ones of the segmented particles, for example, including size and shape related features, a cavings identification module 136′ configured to identify cavings particles as those having at least one size related feature greater than a threshold, and a shape classification module 138′ configured to process the extracted properties and to group each of the identified cavings particles into a corresponding shape classification or category (as described in more detail below with respect to FIG. 5). A database manager 140′ may be in communication with onboard memory 142 to temporarily store the images, segmented images, extracted properties, and classification data as well as with an external database 150 as described above.

It will be appreciated that system 100′ may be advantageously utilized in remote locations with limited mobile communication networks or other access to larger computer networks including an external backend. In such implementations, digital images may be acquired and evaluated remotely. The images and evaluation results may then be updated to the external database at a later time (or another location having more robust mobile or wireless communications). On the other hand, system 100 may be advantageously utilized in locations having robust mobile networks or other high bandwidth communication channels in which acquired images may be rapidly uploaded to and downloaded from a backend server having more robust processing capabilities.

With continued reference to FIG. 2, it will be appreciated that modules 120, 120′ of systems 100, 100′ may include computer hardware and software configured to automatically or semi-automatically evaluate images of the rock particles. To perform these functions, the hardware may include one or more processors (e.g., microprocessors) which may be connected to one or more data storage devices (e.g., hard drives or solid state memory).

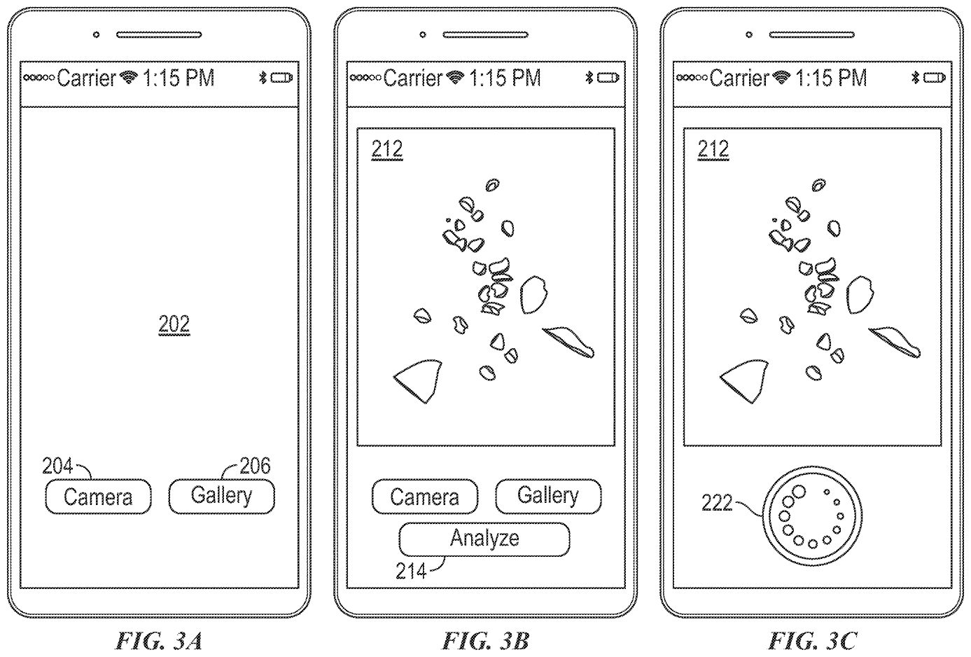

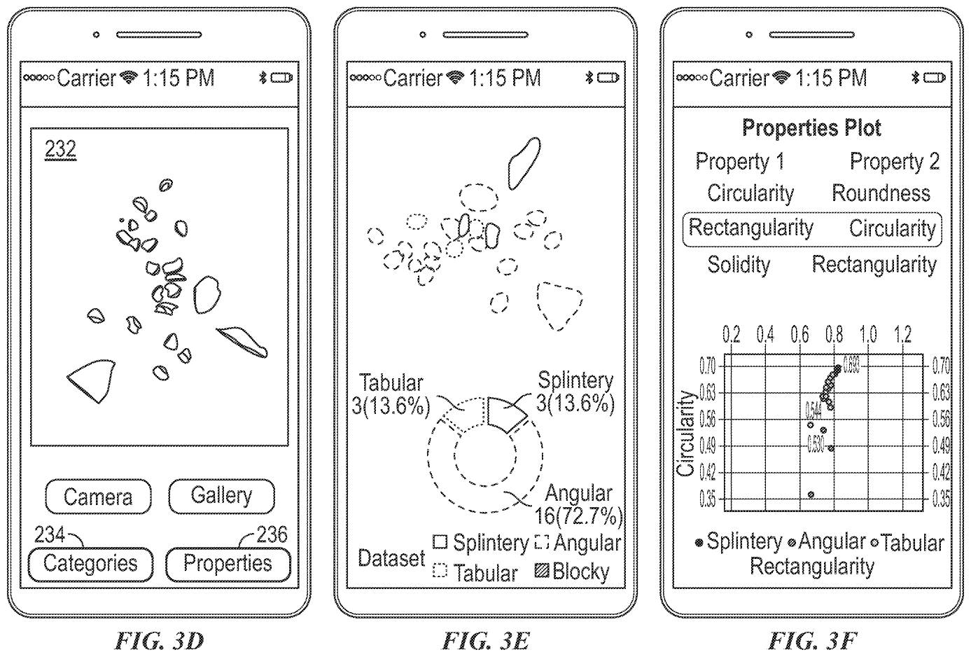

With continued reference to FIG. 2 and further reference to FIGS. 3A-3F (collectively FIG. 3), the mobile device 110 may run a software App configured to enable a user to acquire a digital image of rock particles and upload the image for subsequent processing. When launched, the App may be configured to display a homepage 202 (FIG. 3A), which allows the user to take a digital image of prepared rock particles using the camera tab 204 or to load a pre-existing image using the gallery tab 206. Upon taking or selecting a digital image 212 (as shown in FIG. 3B), the user may click the analyze tab 214 to load the image to a panel and to send it to the processing module for analysis. The App may indicate that the image is uploading and/or processing as shown at 222 in FIG. 3C. When the analysis is completed, the results may be automatically saved to the memory 142 and/or database 150 and displayed on the App (e.g., received or downloaded one of modules 120, 120′ and displayed).

FIG. 3D depicts an example segmented image 232 downloaded to the App. Note that each of the cavings particles is outlined using a polyline. Although not shown, the outline may be colored to indicate the different particle shapes, for example, a first color indicating a first shape classification, a second color indicating a second shape classification, and so on. The App may be further configured to enable the user to display a particle shape classification summary by clicking a categories tab 234. The particle shape classification summary may be in listing or chart form, for example, as a pie chart as shown on FIG. 3E. In the depicted example the summary shows the number of particles in each shape classification. In the depicted embodiment, three of the 22 particles in the segmented image are classified as tabular, three are classified as splintery, and 16 are classified as angular. The App may be still further configured to enable the user to display the particle properties (e.g., the size and shape related features) by clicking a properties tab 236. The properties may be shown, for example, as a two-dimensional scatter plot (a cross plot) of selected properties, for example, plotting particle circularity versus particle rectangularity as shown on FIG. 3F. The disclosed embodiments are, of course, not limited to displaying any particular properties or to showing the properties on a cross plot, but may instead include a property listing, a histogram, or any other suitable display. Moreover, the individual particle instances (segmented particles) may be shown in the display to provide an indication of the particle properties of each of the particles in the image.

As described above with respect to FIG. 3A, the App may be configured to prompt the user to take a digital image of prepared rock particles. It will be appreciated that a mobile device is not generally configured for calibrated image acquisition and that the acquired images may be non-calibrated. Therefore, the App may be further configured to provide instructions or a tutorial of an image acquisition procedure to improve image quality. For example, the user may be instructed to deploy the rock particles on a tray or a paper background having a specific color. The App may further instruct the user to optimize external lighting or may also automatically employ the camera flash to ensure consistent image lighting. The App may be still further configured to instruct the user to hold the mobile device at a particular distance (or within a range of distances) from the particles. Moreover, in mobile devices including a depth sensor (e.g., an optical depth sensor such as Lidar), the mobile device may be configured to instruct the user to move the mobile device closer to or further from the particles until the distance between the mobile device and the rocks particles is within a predetermined range. The App may also be configured to disable the camera unless it is within the predetermined range of distances.

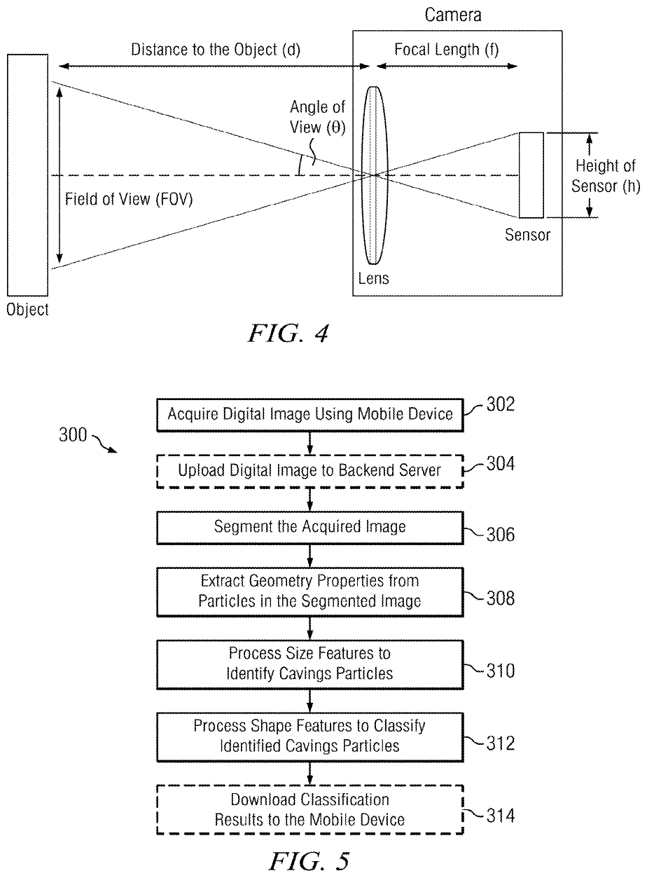

In certain applications, it may be important to measure size features (e.g., the length, width, and/or area) of the particles when extracting geometry properties. These size features may be measured, for example, based on the size of an individual pixel in the image. The pixel size may be determined, for example, using an integrated depth sensor (e.g., a Lidar sensor) in the mobile device or by including a known reference object in the image. In mobile device embodiments including a depth sensor, the App may be configured to access the depth sensor when taking an image to measure the distance between the camera and the particles. With reference to FIG. 4, the field of view FOV of the image may be computed from the measured distance d and the known focal length f and sensor height h (size) of the camera or from the angle of view θ of the camera, for example, as follows:

$$FOV = 2 d tan θ$$

$$θ = 2 arctan \frac{h}{2 f}$$

The pixel size may then be determined by dividing the field of view by the number of pixels along the height of the camera sensor N, for example, as follows:

$$PixelSize = \frac{FOV}{N}$$

It will be appreciated that the pixel sizing routine may be employed to determine a global pixel size (a pixel size across the whole image) or a local pixel size (e.g., a pixel size for an individual particle in the image). The primary difference between a global pixel size and a local pixel size is in the distance measurement. A global pixel size calculation may use an average distance or a distance at a central location in the image while a local pixel size calculation may use distinct distance measurements for each individual particle obtained, for example, from a Lidar depth map.

In other embodiments, the App may be configured to determine the pixel size from a known sizing object in the image. In such embodiments, the App may instruct the user to include a sizing object in the field of view and further query the user to enter the size of the object (e.g., the diameter or other dimension). This sizing information may then be transferred or uploaded to the processing module 120, 120′ along with the image. The processing module may also be configured to segment and identify the sizing object (e.g., in the case of a coin the object may be segmented and then identified as having a circularity greater than a threshold). The pixel size may be determined from the known size of the identified object (e.g., by dividing the known diameter of a coin by the number of pixels along the diameter of the identified coin). It will be appreciated that the image may include multiple sizing objects (e.g., one in each corner of the image or one at an upper end and another at a lower end of the image) to compensate for image distortion or inclination of the camera with respect to the image plane. Moreover, in some embodiments, the backend may be configured to recognize various standard objects such as various coins or a ruler (e.g., using a machine learning image recognition routine) and determine the pixel size from the known size of the identified object (e.g., the known diameter of an identified coin).

In certain embodiments, the App may be further configured to check the orientation of the mobile device prior to taking a digital image. For example, the App may be configured to access and process accelerometer measurements made in the device to determine the orientation of the device with respect to horizontal. The App may be configured to show a warning light on the user interface or even disable the camera when a deviation from horizontal exceeds a threshold (e.g., 10 degrees). Moreover, the App may be further configured to instruct the user about which axis to rotate the device to achieve an acceptable horizontal orientation.

Turning now to FIG. 5, a flow chart of an example method 300 for evaluating rock particles is depicted. Method 300 includes acquiring a digital image of rock particles separated from drilling fluid using a handheld mobile device at 302. The particles may include cuttings and/or cavings particles generated, for example, while drilling a wellbore and may be transported to the surface in circulating drilling fluid as described above with respect to FIG. 1. In example embodiments, the digital image may be uploaded via a mobile or wireless network to a backend server at 304, for example, as described above with respect to FIGS. 2 and 3. The acquired image may be processed with a segmenting module to obtain a segmented image at 306. The segmenting module may be configured, for example, to identify a plurality of individual particles in the uploaded image. The segmented image may be processed at 308 to extract geometry properties from each of a plurality of selected particles in the segmented image. For example, the geometry properties may be extracted from each of the segmented particles or a subset of the segmented particles. The selected particles may be evaluated particle by particle, for example, to extract the geometry properties (e.g., the size and shape related features).

In one example embodiment the geometry properties may include size and shape related features. The size related features may be evaluated at 310 to identify cavings particles among the segmented particles (e.g., distinguish between cuttings and cavings particles). For example, the identification may be based on one or more size related feature threshold(s) in which cuttings particles are identified and classified as having a size feature less than the threshold and cavings particles are identified and classified as having a size feature greater than the threshold. In one example embodiment, cavings particles may be identified as those having a diameter (e.g., a maximum diameter or average diameter) greater than a diameter threshold (e.g., greater than about 3 mm). In other embodiments, the cavings identification may make use of particle area, perimeter, volume, or other thresholds.

The shape related features may be processed at 312 to classify each of the identified cavings particles with a corresponding particle shape (e.g., and thereby generate a particle shape classification). In example embodiments, the segmented image, the extracted properties, and the shape classification may then be automatically downloaded to the handheld mobile device at 314 where the results may be displayed to the user. In embodiments in which the backend modules are employed on the mobile device as depicted on FIG. 2B, the segmented image, the extracted properties, and the shape classification may be automatically displayed upon completion of the processing.

With continued reference to FIG. 5, it will be appreciated that method 300 may advantageously enable automated (or semi-automated) classification of rock particle shape without (or with limited) human intervention. For example, human input to the method may be limited to taking an image and uploading the image to the backend server at 302 and 304. Segmenting the image at 306, extracting the geometry properties at 308, identifying cavings particles at 310, classifying the cavings particles at 312, and downloading the segmented image, the extracted properties, and the particle classification to the mobile device at 312 may be performed automatically without human intervention.

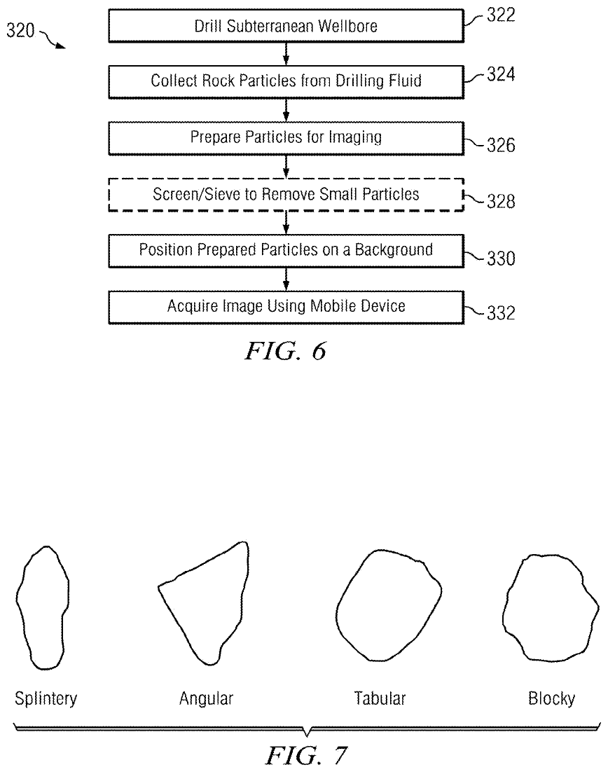

FIG. 6 depicts one example method 320 for separating the rock particles from the drilling fluid and acquiring a digital image at 302 of FIG. 5. A borehole is drilled through a subterranean formation of interest at 322, for example, using the example rig 20 described above with respect to FIG. 1. Rock cuttings and cavings particles are transported to the surface in the upwardly flowing drilling fluid (at 94 in FIG. 1). These particles may be collected at 324, for example, using a shale shaker or other solids separation/control equipment. The particles obtained from the shale shaker may be prepared for image analysis at 326, for example, by washing and then drying in an oven. The particles may further be optionally screened or sieved to remove small or large particles at 328. In example embodiments, the particles may be screened to retain primarily cavings particles. In other embodiments the sampled particles may include both cuttings and cavings particles. It will be appreciated that cuttings and cavings particles are commonly distinguished in the industry based on size, with particles having a size less than a threshold being classified as cuttings and particles having a size greater than the threshold being classified as cavings.

The prepared rock particles may be placed in a tray or on a sheet of paper at 330 as described above with respect to FIG. 2. In example embodiments, the tray or payer may have a predetermined color, for example, a high contrast (vivid) background color to enhance subsequent particle identification and segmentation in the acquired images, for example, pure magenta (e.g., with RGB values of 255, 0, 255), pure blue (e.g., with RGB values of 0, 0, 255), pure green (e.g., with RGB values of 0, 255, 0), and so forth. In general, such colors do not exist in nature and, accordingly, help instance segmentation models avoid detecting the background of the tray as part of the particle. The tray may be placed on a table or other horizontal surface where a digital image may be taken at 332 using a mobile device as described above. It will be appreciated that the image may be taken under non-calibrated lighting conditions (e.g., in ambient light or using the camera flash as also described above) or under calibrated lighting conditions that make use of external lighting and light sensors.

With reference again to FIG. 5, in example embodiments, the segmenting module may employ a Mask Region-Based Convolutional Neural Network (Mask R-CNN) such as disclosed in U.S. patent application Ser. No. 17/647,407, which is incorporated by reference herein in its entirety. The Mask R-CNN may be configured to identify the individual particles in the digital images and thereby generate the segmented image at 306. For example, the Mask R-CNN may produce, for example, bounding boxes and mask images. The bounding boxes may be defined as a set of x-y coordinates in an image that indicates an image region that contains an object of interest. The bounding box may include a confidence score that ranges from 0 to 1 (e.g., with greater values indicating higher confidence regarding) for each object of interest. The mask image may indicate (e.g., highlight or otherwise bound) regions of interest that have a confidence score that exceeds a threshold.

It will be appreciated that Mask R-CNN is a model architecture that falls in the supervised learning category, meaning that it commonly requires a training dataset that includes images and corresponding labels. For example, the model may be trained using images including cuttings and/or cavings particles of various sizes, shapes, colors, and lithology types. It will be further appreciated that the R-CNN model may be continuously retrained during a drilling operation or between drilling operations in a field. For example, segmentation errors may be identified and corrected and then used to generate labeled training images that may be used to retrain (or further train) the R-CNN.

In example embodiments, a segmented image may depict a plurality of rock particles on a high contrast tray. The individual particles may be identified, for example, via a particle outline or other demarcation. Moreover, each identified particle may be identified by a corresponding set of pixels in the image. Stated another way the segmented image may include a pixel by pixel segmentation in which each pixel in the image is assigned to the background or to a single individual particle.

In some operations, cavings particles may be placed on a tray or holder having a black (or dark colored) background grid prior to imaging. It will be appreciated that such a grid may at times interfere with image segmentation. The segmentation procedure may be modified to accommodate the grid. For example, in one approach, image processing techniques, such as morphological closing, may be used to remove the grid from the images prior to segmentation. In another approach, the segmentation model may be trained using images in which the cuttings and/or cavings particles are placed on a holder including a black (or other dark colored) grid (as opposed to a background having a vivid color). The training may include images having grids of multiple sizes and colors. Image segmentation at 306 may then use the grid trained segmentation model.

It may be advantageous to employ different segmentation models at 306 depending on whether or not the images include a background grid. For example, images that do not include a background grid may employ a segmentation model trained using images that do not include a grid while images that include a background grid may employ a segmentation model trained using images that include background grid. In some embodiments, the processing at 306 may further include evaluating the image to determine whether or not the image includes a background grid and then selecting the appropriate segmentation model based the evaluation. The disclosed embodiments are, of course, not limited in this regard.

With further reference to FIG. 5, the geometry properties extracted at 308 may include substantially any size related features and/or shape related features of the particle. For example, the extracted geometry properties may include at least one size related feature such as an equivalent particle diameter, an area, a perimeter, a maximum axis, a minimum axis, and a particle aspect ratio (maximum axis to minimum axis ratio). These size related features may be extracted, for example, via pixel measurements. For example, the particle area may include the number of pixels in the cross section of the particle. Likewise, the maximum and minimum axes and the perimeter may be defined as a number of pixels along the axis. These features may also be converted to conventional metric dimensions based on the computed pixel size. Moreover, the geometry related properties may further include spatial relationships of the pixels grouped in each particle to extract particle roundness, circularity, rectangularity, solidity, elongation, eccentricity, compactness, convexity, curl, orientation, and/or convex hull area. It will be appreciated that these shape related features are well defined in digital image processing applications, for example, as defined in Open CV (https://docs.opencv.org/3.4/d3/dc0/group imgproc shape.html), ImageJ User Guide (https://imagej.nih.gov/ij/docs/guide/user-guide.pdf), and Shape Analysis and Measurement (http://www.cyto.purdue.e w/cdroms/micro2/content/education/wirth 10.pdf).

With still further reference to FIG. 5, extracted size related features may be processed at 310 to identify cavings particles in the image. As described above, the identification may include comparing extracted size features for each segmented particle with one or more size feature thresholds. Particles that exceed the threshold(s) may be identified as cavings particles and the remainder may be identified as cuttings particles. As described above, the size related features may include particle diameter, perimeter, area, and the like.

The extracted shape related features may be processed at 312 using the shape classification module 138, 138′ (FIG. 2) to classify the identified cavings particles with a corresponding particle shape. The shape classification may include substantially any suitable shape, however, cavings particles are commonly classified into four distinct categories; splintery, angular, tabular, and blocky. FIG. 7 depicts examples of these four cavings particle shape classifications.

It will be appreciated that the shape of cavings particles is sometimes used as an indicator of a formation failure mechanism. For example, splintery cavings may be taken as an indication of a tensile failure while angular cavings may be taken as an indication of shear failure. Likewise, tabular cavings may be taken as an indication of slip failure caused by bedding planes while blocky cavings may be taken as an indicator that the formation failure is caused by pre-existing fractures. Moreover, a group of cavings particles having a mixture of these shapes may indicate multiple simultaneous failure mechanisms. Advantageous embodiments may classify each of the particles as being one of these four categories and may further classify or summarize the amount or percentage of each shape in a group of particles (e.g., collected in one or more images).

The particle shape may be assigned, for example, based on any one or more of the above listed geometry properties. In one embodiment, the shape classification may employ a heuristic approach in which thresholds are applied to computed geometric properties in a hierarchical manner. In one example heuristic methodology, particles that exceed a first threshold of a first property may be assigned to first classification. Among the remaining particles (those that do not exceed the first threshold), those that exceed a second threshold of a second property may be assigned to second classification. Among the particles that do not exceed either the first or the second thresholds, those that exceed a third threshold of a third property may be assigned to a third classification. The remaining particles (that do not exceed at least one of the first, second, and third thresholds) may be assigned to a fourth classification. For example only, a cavings particle may be classified as splintery when an elongation feature and/or an aspect ratio feature is greater than a threshold (indicating that the maximum axial dimension of the particle is significantly greater than the minimum axial dimension). Among the remaining (non-splintery) particles, those that have a rectangularity feature that exceeds a threshold may be classified as tabular. Those particles that are neither spintery nor tabular that have a circularity feature that exceeds a threshold may be classified as blocky. The remaining particles not meeting any of these criteria may be classified as angular.

In another embodiment, the cavings particle may be classified according to a “location” of the particle in a multi-dimensional space of extracted geometry properties. As described above, a set of geometry properties may be computed (e.g., for each of the selected particles). The set of geometry properties may include substantially any number of size and shape related features, for example, including at least 4 features (e.g., at least 6, 8, 12, or 16 features). The shape classification module may be configured to classify the particle according to values of those geometry properties, for example, that cause like particles to cluster in the aforementioned multi-dimensional feature space. The particle may alternatively (and/or additionally) be classified based on a nearest neighbor classification of the particle in the multi-dimensional space of extracted color and texture features. In example embodiments a classification of each of the particles may be assigned based on the clustering. In such an embodiment, groups of particles located in the same cluster (or local region of the hyperspace) may be assigned the same classification.

In still other embodiments, the shape classification module may be configured to use a trained machine learning model. In such embodiments, a classification model may be trained using geometry properties that are extracted from labeled cavings particles. Once trained, the model may be used to infer the cavings shape classification from a segmented image and corresponding extracted geometry properties.

With continued reference to FIG. 5, the extracted size related features may be further processed at 310 and/or 312 to classify the size of the identified cavings particles. For example, each identified cavings particle may be classified as a small, medium, or large cavings particle (e.g., in addition to having splintery, tabular, blocky, or angular shape). The particle size classification may make use of size related features such as the equivalent particle diameter, an area, a perimeter, a maximum axis, a minimum axis, a sum of the maximum and minimum axis, or other similar size related features. For example, the particle size maybe classified by comparing one or more size related features with corresponding thresholds. In one embodiment, a rock particle may be classified as a large cavings particle when a first size related feature (such as equivalent particle diameter) exceeds a first threshold. The particle may be classified as a small cavings particle when the first size related feature is less than a second threshold and as a medium cavings particle when the first size related feature is greater than the second threshold but less than the first threshold. Advantageous embodiments may further classify or summarize the amount or percentage of particles in each size classification.

As noted above, the particle size and shape classification and/or summary may provide the user with an indication of formation failure mechanisms and the overall integrity and/or stability of the wellbore. In certain embodiments, the App may be further configured to highlight certain cavings size and/or shape combinations that are strong indicators of an unstable wellbore or to highlight images having a large percentage of large cavings particles.

With reference again to FIG. 2, the system output (the images, the segmented images, the geometry properties, and the particle classifications) may be stored in one or more databases 150, 170. The database may be configured to include multiple types of data including numerical data (such as geometry properties), string data (such as user and well name), and binary data (such as image and segmented image). Metadata and output may be sorted in table format, for example as JSON and image data may be stored in a folder system. In another embodiment, an SQL database may be employed with blob storage such as Amazon S3 instead of folder system.

The database may be linked with a cavings dashboard 160 that provides a web-based visualization of the database. Data from the database may be accessed in the dashboard using substantially any connected device, including the mobile device and App. The data in the dashboard by be updated to include the latest data in the database, for example, by clicking “Refresh”. The dashboard interface may be presented in a table format with the columns that are similar to the ones in the database and may include clickable hyperlinks to download the input image, the segmented image, and other processed data such as the particle classification and the particle properties.

It will be appreciated that the disclosed embodiments are not limited to classifying only cavings particles. On the contrary, the disclosed system and method may be configured to evaluate substantially any rock particles acquired during drilling (such as cuttings and/or cavings particles) and may further be configured to distinguish between cuttings and cavings particles (e.g., based on a particle size threshold or as having a distinct non-cavings shape classification). Moreover, the disclosed embodiments may be further configured to classify the cuttings and/or cavings particles according to formation lithology, porosity, and substantially any other formation property or descriptor that may be derived from digital images.

In example embodiments, the processing module may further include a color and texture feature extraction module that is configured to extract color and texture related features from the identified particles. Example color related features may include average (such as mean, median, or mode) red, green, and blue intensities or distributions of or standard deviations of red, green, and blue intensities and/or an average luminance of each particle. The color related features may further include a histogram, a variance, a skewness, and/or a kurtosis of the red, green, and blue intensities. Extracted texture related may quantify various spatial relationships and/or directional changes in pixel color and/or brightness in each particle. Extracted texture related features may include, for example, edge detection, pixel to pixel contrast, correlation, and/or entropy. In addition, in certain embodiments, texture related features may be extracted with techniques such as image texture filters (e.g., Gabor filters, and so forth), an autoencoder, or other deep learning based techniques. Moreover, directional changes may be evaluated, for example, for symmetry and used to generate spectra that may be further compared with reference spectra to assign a texture classification to each particle, such as homogeneous, heterogeneous, grainy, laminate, etc. The texture features may be further evaluated, for example, to characterize the grain size or grain size distribution of grains in the formation or rock particles. For example, the grain size may be identified as fine, medium, coarse or as having an average size and size distribution.

It will be appreciated that the color, texture, and geometry feature extraction may make use of a trained machine learning algorithm or any other deep learning algorithm. Such an algorithm may be trained, for example, using extracted color and texture features of different particle types (e.g., lithology types), sizes, shapes, colors, etc. and may make use of an image database including images of rock particles. Such a database may be maintained, for example, on the backend or may be accessible to the backend modules.

Extracted color and/or texture features may be optionally processed to further classify the rock particles with a corresponding lithology type, for example, as described in U.S. patent application Ser. No. 17/647,412, which is incorporated by reference herein in its entirety. In general, the lithology of a rock particle is a description of its physical characteristics visible at an outcropping, in hand or core samples, or with low magnification microscopy. Lithology may refer to either a detailed description of these physical characteristics, or a summary of the gross physical character of a rock. In a second sense, the lithology of a rock refers to a type of rock or a gross (or macro) identification or classification of the rock. Example lithologies or lithology types in this second sense include sandstone, limestone, slate, shale, basalt, coal, anhydrite, dolomite, gypsum, clay, chert, granite, and the like. As such, color and/or texture features represent physical characteristics of a lithology that may be measured and quantified from a digital image and evaluated to determine the lithology of a formation (in the second sense). For example, the color and/or texture features extracted from selected particles in the segmented image may be evaluated using a trained model to classify the lithology type(s) of those selected particles.

The extracted color and texture features may be still further processed to estimate a formation porosity, for example, according to a “location” of the particle in a multi-dimensional space of extracted color and texture features. For example, a set of color and texture features may be computed (e.g., for each of the selected rock particles). The set of computed color and texture features may include a large number of features, for example, including at 16 or more features. A backend module may be configured to correlate the color and texture features with formation porosity such that in practice the module assigns a porosity value to a rock particle based on the set of values of those features (or stated another way based on the location of the rock in the aforementioned multi-dimensional color/texture feature space).

It will be understood that the present disclosure includes numerous embodiments. These embodiments include, but are not limited to, the following embodiments.

In a first embodiment, a method for classifying a shape of cavings particles includes using a mobile device to acquire a digital image of rock particles generated while drilling a subterranean wellbore; processing the digital image to generate a segmented image that identifies individual ones of the rock particles; extracting a plurality of geometry properties from selected ones of the identified rock particles depicted in the segmented image, the plurality of geometry properties including size features and shape features; evaluating at least one of the size features to identify cavings particles among the identified rock particles; and generating a cavings particle classification by processing a plurality of the shape features to label each of the identified cavings particles with a corresponding cavings shape.

A second embodiment may include the first embodiment, wherein the processing the digital image to generate a segmented image, the extracting a plurality of geometry properties, the evaluating at least one of the size features, and the processing the plurality of the shape features are performed by the mobile device.

A third embodiment may include the first embodiments, wherein the processing the digital image to generate a segmented image, the extracting a plurality of geometry properties, the evaluating at least one of the size features, and the processing the plurality of the shape features are performed by a processor that is external to the mobile device; and the method further comprises uploading the digital image from the mobile device to the external processor and downloading the cavings particle shape classification from the processing module to the mobile device.

A fourth embodiment may include any one of the first through the third embodiments, wherein the acquiring the digital image comprises: drilling a subterranean wellbore; collecting the rock particles from circulating drilling fluid; preparing the rock particles for imaging; and using the mobile device to take a digital photograph of the rock particles.

A fifth embodiment may include any one of the first through the fourth embodiments, wherein the evaluating at least one of the size features comprises identifying and labeling the identified rock particle as a cavings particle when the at least one of the size features is greater than a corresponding size feature threshold.

A sixth embodiment may include any one of the first through the fifth embodiments, wherein the processing the plurality of shape features employs a heuristic approach in which thresholds are applied to the extracted shape features in a hierarchical manner.

A seventh embodiment may include the sixth embodiment, wherein the processing the plurality of shape features comprises: assigning the cavings particles that exceed a first shape feature threshold to a first shape classification; assigning the cavings particles that exceed a second shape feature threshold and do not exceed the first shape feature threshold to a second shape classification; assigning the cavings particles that exceed a third shape feature threshold and do not exceed the first shape feature threshold or the second shape feature threshold to a third shape classification; and assigning the cavings particles that do not exceed the first shape feature threshold, the second shape feature threshold, or the third shape feature threshold to a fourth shape classification.

An eighth embodiment may include the seventh embodiment, wherein: the first shape feature threshold is an elongation threshold; the second shape feature threshold is a rectangularity threshold; and the third shape feature threshold is a circularity threshold.

A ninth embodiment may include any one of the first through the eighth embodiments, wherein the evaluating at least one of the size features further comprises labelling each of the identified cavings particles with a corresponding size.

A tenth embodiment may include any one of the first through the ninth embodiments, wherein the cavings particle classification comprises the segmented image, the extracted geometry properties, and a shape classification summary.

In an eleventh embodiment an integrated mobile system for evaluating cavings particles includes a mobile device including a digital camera configured to acquire a digital image of rock particles generated while drilling a subterranean wellbore; and a processing module configured to receive the digital image from the digital camera, the processing module including an image segmentation module configured to process the digital image and generate a segmented image, a geometry properties extraction module configured to extract size features and shape features from selected ones of the rock particles in the segmented image, a cavings identification module configured to process at least one of the size features to identify cavings particles among the rock particles, and a shape classification module configured to generate a cavings particle shape classification by processing a plurality of the shape features to label each of the identified cavings particles with a corresponding cavings shape.

A twelfth embodiment may include the eleventh embodiment, wherein the processing module is in the mobile device.

A thirteenth embodiment may include the eleventh embodiment, wherein the processing module is external to the mobile device; the mobile device is configured to upload the digital images to the processing module via a mobile or wireless communication link; and the processing module is configured to download the cavings particle shape classification to the mobile device.

A fourteenth embodiment may include any one of the eleventh through thirteenth embodiments, further comprising: a database manager in communication with a database configured to store the digital image, the segmented image, the extracted geometry properties, and the cavings particle shape classification; and a dashboard configured to provide a web based visualization of the database.

A fifteenth embodiment may include any one of the eleventh through fourteenth embodiments, wherein the shape classification module is configured to process the extracted geometry properties using a heuristic approach in which thresholds are applied to the extracted shape features in a hierarchical manner such that the identified cavings particles that exceed a first shape feature threshold are assigned to a first shape classification, the identified cavings particles that exceed a second shape feature threshold and do not exceed the first shape feature threshold are assigned to a second shape classification, the identified cavings particles that exceed a third shape feature threshold and do not exceed the first shape feature threshold or the second shape feature threshold are assigned to a third shape classification, and the identified cavings particles that do not exceed the first shape feature threshold, the second shape feature threshold, or the third shape feature threshold are assigned to a fourth shape classification.

In a sixteenth embodiment, a method for classifying a shape of cavings particles generated will drilling a subterranean wellbore includes using a mobile device to acquire a digital image including both cuttings particles and cavings particles generated while drilling the subterranean wellbore; processing the acquired digital image to generate a segmented image that identifies individual ones of the cuttings particles and the cavings particles depicted in the digital image; extracting size features and shape features from the segmented cuttings particles and the segmented cavings particles in the segmented image; processing at least one of the extracted size features to identify the cavings particles; and generating a cavings particle shape classification by processing a plurality of the shape features to label each of the identified cavings particles with a corresponding cavings shape.

A seventeenth embodiment may include the sixteenth embodiment, wherein: the mobile device further measures a distance between the mobile device and the cuttings particles and cavings particles; the extracted size features are in pixel units; and processing the at least one of the extracted size features further comprises (i) processing the distance to compute a pixel size in the digital image, (ii) multiplying the pixel size by the at least one of the extracted size features to obtain a particle size, and (iii) comparing the particle size with a particle size threshold to identify the cavings particles.

An eighteenth embodiment may include the sixteenth embodiment, wherein the digital image further includes a sizing object therein; the extracted size features are in pixel units; and processing the at least one of the extracted size features further comprises (i) determining a pixel size of the sizing object, (ii) dividing a known size of the sizing object by the pixel size of the sizing object to obtain a pixel size in the digital image, (iii) multiplying the pixel size by the at least one of the extracted size features to obtain a particle size, and (iv) comparing the particle size with a particle size threshold to identify the cavings particles.

A nineteenth embodiment may include any one of the sixteenth through eighteenth embodiments, wherein the processing the plurality of shape features employs a heuristic approach in which thresholds are applied to the extracted shape features in a hierarchical manner in which: assigning the cavings particles that exceed a first shape feature threshold to a first shape classification; assigning the cavings particles that exceed a second shape feature threshold and do not exceed the first shape feature threshold to a second shape classification; assigning the cavings particles that exceed a third shape feature threshold and do not exceed the first shape feature threshold or the second shape feature threshold to a third shape classification; and assigning the cavings particles that do not exceed the first shape feature threshold, the second shape feature threshold, or the third shape feature threshold to a fourth shape classification.

A twentieth embodiment may include any one of the sixteenth through nineteenth embodiments, wherein the first shape feature threshold is an elongation threshold; the second shape feature threshold is a rectangularity threshold; and the third shape feature threshold is a circularity threshold.

In a twenty-first embodiment a method for evaluating rock particles includes using a mobile device to acquire a digital image of rock particles generated while drilling a subterranean wellbore; processing the digital image to generate a segmented image that identifies individual ones of the rock particles; extracting a plurality properties from selected ones of the identified rock particles depicted in the segmented image, the plurality of properties including at least one of color features, texture features, size features, and shape features; and evaluating at least one of the plurality of properties to determine at least one of a porosity and a lithology of a formation through which the wellbore penetrates.

Although an integrated mobile system for formation rock analysis has been described in detail, it should be understood that various changes, substitutions and alternations can be made herein without departing from the spirit and scope of the disclosure as defined by the appended claims.

1. A method for classifying a shape of cavings particles, the method comprising:
using a mobile device to acquire a digital image of rock particles generated while drilling a subterranean wellbore;
processing the digital image to generate a segmented image that identifies individual ones of the rock particles;
extracting a plurality of geometry properties from selected ones of the identified rock particles depicted in the segmented image, the plurality of geometry properties including size features and shape features;
evaluating at least one of the size features to identify cavings particles among the identified rock particles; and
generating a cavings particle classification by processing a plurality of the shape features to label each of the identified cavings particles with a corresponding cavings shape.

2. The method of claim 1, wherein the processing the digital image to generate a segmented image, the extracting a plurality of geometry properties, the evaluating at least one of the size features, and the processing the plurality of the shape features are performed by the mobile device.

3. The method of claim 1, wherein
the processing the digital image to generate a segmented image, the extracting a plurality of geometry properties, the evaluating at least one of the size features, and the processing the plurality of the shape features are performed by a processor that is external to the mobile device; and
the method further comprises uploading the digital image from the mobile device to the external processor and downloading the cavings particle shape classification from the processing module to the mobile device.

4. The method of claim 1, wherein the acquiring the digital image comprises:
drilling a subterranean wellbore;
collecting the rock particles from circulating drilling fluid;
preparing the rock particles for imaging; and
using the mobile device to take a digital photograph of the rock particles.

5. The method of claim 1, wherein the evaluating at least one of the size features comprises identifying and labeling the identified rock particle as a cavings particle when the at least one of the size features is greater than a corresponding size feature threshold.

6. The method of claim 1, wherein the processing the plurality of shape features employs a heuristic approach in which thresholds are applied to the extracted shape features in a hierarchical manner.

7. The method of claim 6, wherein the processing the plurality of shape features comprises:
assigning the cavings particles that exceed a first shape feature threshold to a first shape classification;
assigning the cavings particles that exceed a second shape feature threshold and do not exceed the first shape feature threshold to a second shape classification;
assigning the cavings particles that exceed a third shape feature threshold and do not exceed the first shape feature threshold or the second shape feature threshold to a third shape classification; and
assigning the cavings particles that do not exceed the first shape feature threshold, the second shape feature threshold, or the third shape feature threshold to a fourth shape classification.

8. The method of claim 7, wherein:
the first shape feature threshold is an elongation threshold;
the second shape feature threshold is a rectangularity threshold; and
the third shape feature threshold is a circularity threshold.

9. The method of claim 1, wherein the evaluating at least one of the size features further comprises labelling each of the identified cavings particles with a corresponding size.

10. The method of claim 1, wherein the cavings particle classification comprises the segmented image, the extracted geometry properties, and a shape classification summary.

11. An integrated mobile system for evaluating cavings particles, the system comprising:
a mobile device including a digital camera configured to acquire a digital image of rock particles generated while drilling a subterranean wellbore; and
a processing module configured to receive the digital image from the digital camera, the processing module including an image segmentation module configured to process the digital image and generate a segmented image, a geometry properties extraction module configured to extract size features and shape features from selected ones of the rock particles in the segmented image, a cavings identification module configured to process at least one of the size features to identify cavings particles among the rock particles, and a shape classification module configured to generate a cavings particle shape classification by processing a plurality of the shape features to label each of the identified cavings particles with a corresponding cavings shape.

12. The system of claim 11, wherein the processing module is in the mobile device.

13. The system of claim 11, wherein:
the processing module is external to the mobile device;
the mobile device is configured to upload the digital images to the processing module via a mobile or wireless communication link; and
the processing module is configured to download the cavings particle shape classification to the mobile device.

14. The system of claim 11, further comprising:
a database manager in communication with a database configured to store the digital image, the segmented image, the extracted geometry properties, and the cavings particle shape classification; and
a dashboard configured to provide a web based visualization of the database.

15. The system of claim 11, wherein the shape classification module is configured to process the extracted geometry properties using a heuristic approach in which thresholds are applied to the extracted shape features in a hierarchical manner such that the identified cavings particles that exceed a first shape feature threshold are assigned to a first shape classification, the identified cavings particles that exceed a second shape feature threshold and do not exceed the first shape feature threshold are assigned to a second shape classification, the identified cavings particles that exceed a third shape feature threshold and do not exceed the first shape feature threshold or the second shape feature threshold are assigned to a third shape classification, and the identified cavings particles that do not exceed the first shape feature threshold, the second shape feature threshold, or the third shape feature threshold are assigned to a fourth shape classification.

16. A method for classifying a shape of cavings particles generated will drilling a subterranean wellbore, the method comprising:
using a mobile device to acquire a digital image including both cuttings particles and cavings particles generated while drilling the subterranean wellbore;
processing the acquired digital image to generate a segmented image that identifies individual ones of the cuttings particles and the cavings particles depicted in the digital image;
extracting size features and shape features from the segmented cuttings particles and the segmented cavings particles in the segmented image;
processing at least one of the extracted size features to identify the cavings particles; and
generating a cavings particle shape classification by processing a plurality of the shape features to label each of the identified cavings particles with a corresponding cavings shape.

17. The method of claim 16, wherein:
the mobile device further measures a distance between the mobile device and the cuttings particles and cavings particles;
the extracted size features are in pixel units; and
processing the at least one of the extracted size features further comprises (i) processing the distance to compute a pixel size in the digital image, (ii) multiplying the pixel size by the at least one of the extracted size features to obtain a particle size, and (iii) comparing the particle size with a particle size threshold to identify the cavings particles.

18. The method of claim 16, wherein
the digital image further includes a sizing object therein;
the extracted size features are in pixel units; and
processing the at least one of the extracted size features further comprises (i) determining a pixel size of the sizing object, (ii) dividing a known size of the sizing object by the pixel size of the sizing object to obtain a pixel size in the digital image, (iii) multiplying the pixel size by the at least one of the extracted size features to obtain a particle size, and (iv) comparing the particle size with a particle size threshold to identify the cavings particles.

19. The method of claim 16, wherein the processing the plurality of shape features employs a heuristic approach in which thresholds are applied to the extracted shape features in a hierarchical manner in which:
assigning the cavings particles that exceed a first shape feature threshold to a first shape classification;
assigning the cavings particles that exceed a second shape feature threshold and do not exceed the first shape feature threshold to a second shape classification;
assigning the cavings particles that exceed a third shape feature threshold and do not exceed the first shape feature threshold or the second shape feature threshold to a third shape classification; and
assigning the cavings particles that do not exceed the first shape feature threshold, the second shape feature threshold, or the third shape feature threshold to a fourth shape classification.

20. The method of claim 19, wherein:
the first shape feature threshold is an elongation threshold;
the second shape feature threshold is a rectangularity threshold; and
the third shape feature threshold is a circularity threshold.
